(19) United States
(12) Patent Application Publication
(10) Pub. No.: US 2005/0095259 A1
(43) Pub. Date: May 5, 2005

(54) Manufacturing method for entomopathogenic fungi

(76) Inventor: Chien Yi Liu, Shulin City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/699,877

(22) Filed: Nov. 4, 2003

(51) Int. Cl. A61K 35/84; C12N 1/16

(52) U.S. Cl. 424/195.15; 435/254.1

(57) ABSTRACT

A manufacturing method for extomopathogenic fungi includes the following steps. Step A (drying): the cooked rice with the spores of metarhizium anisopliae is dried in a dry in a certain temperature to make the spores have a certain hydrous rate, and step B (sieving): the dried rice is stirred and some powder is added into the dried rice for the spores being easily detached from the dried rice and the dried rice is sieved from the spores and the powder.

### BACKGROUND OF THE INVENTION

1. Field of the Invention

The present invention relates to manufacturing method for entomopathogenic fungi, and more particularly to a manufacturing method for extomopathogenic fungi and extending the preservation time limit of the entomopathogenic fungi.

2. Description of Related Art

A chemical agent is usually used as a method for a blight prevention and cure of plants. However, the chemical agent may pollute the natural environment. Consequently, a biological agent gradually replaces the chemical agent.

The most popular biological agent is the entomopathogenic fungus, especially metarhizium anisopliae. The quantity of the hosts of the metarhizium anisopliae is over two hundreds so that the metarhizium anisopliae is widely used the blight prevention and cure of plants. The metarhizium anisopliaes forms multiple spores on the exterior of the host after parasitism. The spores germinate and form hyphas when meet a suitable environment. The spore also secretes enzyme to decompose the exterior of the host to make the hyphas smooth go into the body of the host. The hyphas secrete dextruxin to make the host die within three days or the hyphas full fill the host that will die in five or six days. The body of the host is filled with conidia spores that are blackish green and as an infect source of next cycle.

The conventional manufacturing method is to heat the rice with water. The cooked rice is filtered from the water and packed in a plastic bag for disinfecting when the periphery of the rice contains moisture. The cooked rice is kneaded for 10-14 days till the spores distributed all over the outer periphery of the cooked rice. The cooked rice with the spores is kept in a refrigerator. When using the agent. The spores are washed off by using water containing natural detergent. The water with the spores is sprinkled over the soil.

The spore easily sprouts when meeting water so that the conventional keeping method directly keeps the plastic bags in the refrigerator. However, the plastic bags occupies most of the space of the refrigerator and the plastic bags cannot be stored in the refrigerator for a long time because the cooked rice will become moldy when being polluted and the spores may sprout and cannot be used.

The present invention has arisen to mitigate and/or obviate the disadvantages of the conventional manufacturing method for extomopathogenic fungi.

### SUMMARY OF THE INVENTION

The main objective of the present invention is to provide an improved manufacturing method for extomopathogenic fungi and extending the preservation time limit of the entomopathogenic fungi.

To achieve the objective, the manufacturing method for extomopathogenic fungi in accordance with the present invention comprises the following steps. Step A (drying): the cooked rice with the spores of metarhizium anisopliae is dried in a dry in a certain temperature to make the spores have a certain hydrous rate, and step B (sieving): the dried rice is stirred and some powder is added into the dried rice for the spores being easily detached from the dried rice and the dried rice is sieved from the spores and the powder.

Further benefits and advantages of the present invention will become apparent after a careful reading of the detailed description with appropriate reference to the accompanying drawings.

### BRIEF DESCRIPTION OF THE DRAWINGS

FIG. 1 is a flow chart of a manufacturing method for extomopathogenic fungi in accordance with the present invention and extending the preservation time limit of the entomopathogenic fungi.

### DETAILED DESCRIPTION OF THE INVENTION

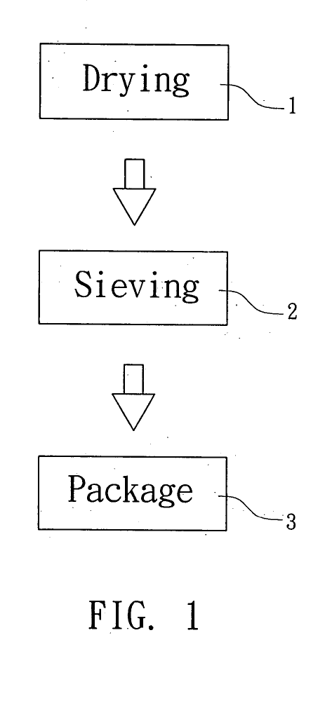

Referring to the drawings and initially to FIG. 1, a manufacturing method for extomopathogenic fungi and extending the preservation time-limit of the entomopathogenic fungi in accordance with the present invention comprises the steps as follow.

Step A (drying): The cooked rice with the spores of metarhizium anisopliae is dried in a dry machine. In the preferred embodiment of the present invention, the dry temperature is set from 15° C. to 25° C. and the spores is dried to have a hydrous rate between 7% to 10%.

Step B (sieving): The dried rice is stirred and some powder is added into the dried rice for the spores being easily detached from the dried rice. Then the dried rice is sieved from the spores and the powder. In the preferred embodiment of the present invention, a shake separator is provided to sieve the dried rice from the spores and the powder and the powder is maltodextrin.

Step C (package): The powder with the spores is packaged into a pack with a suitable volume.

As described above, the cooked rice is sieved so that the volume of the entomopathogenic fungi is greatly reduced when being stored or transported. The spores of the entomopathogenic fungi are dried the hydrous rate thereof is controlled between from 7% to 10% so that the spores do not easily become moist and being polluted. Consequently, the preservation time limit of the entomopathogenic fungi is lengthened.

Furthermore, the powder for detaching the spores from the cooked rice is maltodextrin that is dissolvable. Consequently, the extomopathogenic fungi are more easily spray on the leaves of the plants, the soil or directly embedded into soil.

Although the invention has been explained in relation to its preferred embodiment, it is to be understood that many other possible modifications and variations can be made without departing from the spirit and scope of the invention as hereinafter claimed.

1. A manufacturing method for entomopathogenic fungi with an extended preservation time, comprising the steps of:
drying a predetermined amount of a cooked rice composition comprising a cooked rice containing a predetermined quantity of spores of metarhizium anisopliae at a a predetermined drying temperature that imparts to the spores have a predetermined hydrous rate; and
adding and stirring a predetermined powder to the dried rice composition wherein the spores are readily separable from the cooked rice by sieving the dried rice composition and thereby separating said cooked rice from the spores and the predetermined powder.

2. The manufacturing method for entomopathogenic fungi as claimed in claim 1, wherein the drying temperature is in a range of 15° C. to 25° C. and the a predetermined hydrous rate is between 7% to 10%.

3. The manufacturing method for entomopathogenic fungi as claimed in claim 1, wherein the predetermined powder comprises a water-soluble maltodextrin.

4. The manufacturing method for entomopathogenic fungi as claimed in claim 2, wherein the predetermined powder is a water-soluble maltodextrin.

5. The manufacturing method for entomopathogenic fungi as claimed in claim 2, wherein the predetermined powder with the spores is packaged in a predetermined suitable volume.

6. The manufacturing method for entomopathogenic fungi as claimed in claim 3, wherein the predetermined powder with the spores is packaged in a predetermined suitable volume.

7. The manufacturing method for entomopathogenic fungi as claimed in claim 4, wherein the predetermined powder with the spores is packaged in a predetermined suitable volume.
